(19) United States
(12) Patent Application Publication
(10) Pub. No.: US 2024/0366367 A1
(43) Pub. Date: Nov. 7, 2024

(54) ACTUATION BOLT ASSEMBLY FOR A PROSTHETIC VALVE

(71) Applicant: EDWARDS LIFESCIENCES CORPORATION, Irvine, CA (US)

(72) Inventors: Anatoly Dvorsky, Haifa (IL); Alon Ben-Yosef, Ramot Menashe (IL); Eran Grosu, Kfar-Saba (IL); Nikolai Gurovich, Hadera (IL); Michael Bukin, Pardes Hanna (IL); Ilan Leshecz, Hadera (IL); Joseph Mordechai Leichner, Netanya (IL)

(21) Appl. No.: 18/772,519

(22) Filed: Jul. 15, 2024

Related U.S. Application Data
(63) Continuation of application No. PCT/US2023/010867, filed on Jan. 16, 2023.
(60) Provisional application No. 63/345,571, filed on May 25, 2022.
(60) Provisional application No. 63/301,608, filed on Jan. 21, 2022.

(51) Int. Cl. A61F 2/24

(52) U.S. Cl. CPC A61F 2/2418; A61F 2/243

(57) ABSTRACT

This disclosure is directed to prosthetic heart valves and frames for prosthetic heart valves having actuation assemblies with at least two bolts coupled to each other. The actuation assembly with at least two bolts allow different portions of the actuation assembly to be designed to meet different needs. In one example, the actuation mechanism can include a first bolt coupled end-to-end with a second bolt. The second bolt can have a greater diameter and correspondingly greater mechanical strength than the first bolt. The first bolt can have a lesser diameter suitable for minimizing the thickness of surrounding and adjacent valve frame components. The actuation assembly can also include one or more of a nut, a washer, and a portion designed to attach to components of a delivery system. Also disclosed herein are actuation assemblies including a washer secured by a pin extending through the bolt of the actuation assembly.

### CROSS-REFERENCE TO RELATED APPLICATIONS

This application is a continuation of PCT Application No. PCT/US2023/010867, filed on Jan. 16, 2023, which claims the benefit of U.S. Provisional Patent Application No. 63/301,608, filed on Jan. 21, 2022, and of U.S. Provisional Patent Application No. 63/345,571, filed on May 25, 2022, each of which is incorporated by reference herein in its entirety.

### FIELD

The present disclosure relates to implantable expandable prosthetic heart valves and frame structures for use with expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a delivery capsule of the delivery apparatus so that the prosthetic heart valve can self-expand to its functional size.

Various sorts of mechanical actuators can be used to expand a prosthetic heart valve to its functional size. For example, screw actuators can be rotated relative to various components of the prosthetic heart valve to exert an axial force to cause radial expansion. Screw actuators offer several advantages, including precise control over the radial expansion and/or compression of the prosthetic heart valve, and may not require a separate locking mechanism to prevent the prosthetic heart valve from further expansion or compression after it has been expanded to its functional size. However, prosthetic heart valves with screw actuators are subject to several technical challenges. Particularly, the screw actuators are subject to flexural and compressive stresses when the prosthetic heart valve is retained in a compressed or crimped state. If the actuators do not have sufficient mechanical strength, these stresses can cause bending and buckling of the actuator, among other failure modes that can impair or prevent the prosthetic heart valve from expanding to its functional size. It is also desirable for screw actuators to have a narrow cross section as possible to minimize the overall crimp profile of the prosthetic valve.

Accordingly, there is a need for frames for prosthetic heart valves having actuation mechanisms that are both strong enough to withstand the stresses of being crimped or compressed for implantation, and which have a minimal cross-sectional profile.

### SUMMARY

Disclosed herein are actuation bolt assemblies for prosthetic heart valve frames having two or more bolts connected end-to-end with each other. The disclosed actuation bolt assemblies can include bolts designed with different geometries and/or materials tailored to the specific mechanical demands at various positions relative to the prosthetic heart valve frame, allowing, for example, stronger bolts to be used in regions with higher mechanical stresses and narrower bolts to be used in regions with lower mechanical stresses in the compressed configuration. Additionally, the actuation bolt assemblies disclosed herein can avoid the need to weld or stamp a separate stopper device onto the actuation assembly, which reduces manufacturing complexity and avoids mechanically weakening the stamped or welded portions of the actuation assembly. As such, the actuation assemblies disclosed herein offer superior mechanical strength for resisting the stresses associated with crimping or compressing the frame for implantation, while minimizing the overall crimp profile of the prosthetic heart valve.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can comprise an actuation assembly.

In some examples, the actuation assembly can comprise a first bolt and a second bolt aligned end to end relative to each other, wherein an end portion of the first bolt is coupled to an adjacent end portion of the second bolt.

In some examples, the first bolt has a first diameter and the second bolt has a second diameter.

In some examples, the second diameter is greater than the first diameter.

In some examples, the second bolt comprises a threaded bore in the adjacent end portion of the second bolt, wherein a threaded section of the end portion of the first bolt is tightened into the threaded bore of the second bolt.

In some examples, a head portion of the first bolt is configured to be releasably connected to a component of the delivery apparatus.

In some examples, the frame of the prosthetic heart valve can comprise a plurality of interconnected struts.

In some examples, the frame of the prosthetic heart valve can comprise at least one vertically oriented post with a channel extending therethrough.

In some examples, the first bolt extends through the channel of the at least one vertically oriented post.

In some examples, the actuation assembly further comprises a washer comprising an aperture, wherein the end portion of the first bolt extends through the aperture.

In some examples, the washer is disposed between the second bolt and the vertically oriented post.

In some examples, the at least one vertically oriented post comprises a first vertically oriented post and a second oriented post, the first vertically oriented post comprises a first channel extending therethrough, and the second oriented post comprises a second channel extending therethrough.

In some examples, the first bolt extends through the first channel and the second bolt extends through the second channel.

Certain examples concern a prosthetic valve, comprising a radially expandable frame comprising a plurality of interconnected struts, wherein the frame is radially expandable between a radially compressed state and a radially expanded state. A valvular structure is disposed within frame and configured to regulate the flow of blood through the frame in one direction. At least one an actuation assembly is operatively coupled to the frame, and the actuation assembly comprises first and second bolts aligned end-to-end relative to each other. An end portion of the first bolt is coupled to an adjacent end portion of the second bolt, and the first and second bolts are rotatable together in a first rotational direction to produce radial expansion of the frame from the radially compressed state to the radially expanded state.

Certain examples concern a medical assembly, comprising a prosthetic heart valve comprising a radially expandable frame comprising a plurality of interconnected struts, wherein the frame is radially expandable between a radially compressed state and a radially expanded state. The medical assembly also comprises a valvular structure disposed within frame and configured to regulate the flow of blood through the frame in one direction, at least one an actuation assembly operatively coupled to the frame, and a delivery system configured to be releasably connected to a component of the actuation assembly and to advance the prosthetic heart valve through the vasculature of a patient. The actuation assembly comprises first and second bolts aligned end-to-end relative to each other, wherein an end portion of the first bolt is coupled to an adjacent end portion of the second bolt, and the first and second bolts are rotatably coupled to rotate together in a first rotational direction to produce radial expansion of the frame from the radially compressed state to the radially expanded state. The first bolt has a first diameter and the second bolt has a second diameter, and wherein the first diameter is smaller than the second diameter.

Certain examples concern a prosthetic heart valve, comprising a radially expandable frame having a first end, a second end, and a longitudinal axis extending between the first end and the second end, wherein the frame is radially expandable between a radially compressed state and a radially expanded state. The radially expandable frame also comprises a valvular structure disposed within frame and configured to regulate the flow of blood through the frame in one direction, and at least one actuation assembly operatively coupled to the frame. The frame comprises a plurality of axial posts extending parallel to the longitudinal axis and a plurality of angled struts arranged in circumferentially extending rows of angled struts. The actuation assembly comprises first and second bolts aligned end-to-end relative to each other, wherein an end portion of the first bolt is coupled to an adjacent end portion of the second bolt, wherein the first and second bolts are rotatable together in a first rotational direction to produce radial expansion of the frame from the radially compressed state to the radially expanded state, and wherein the first bolt extends through a channel in a first axial post of the plurality of axial posts.

Certain examples concern a prosthetic heart valve, comprising a radially expandable frame comprising a plurality of interconnected struts, wherein the frame is radially expandable between a radially compressed state and a radially expanded state. The prosthetic heart valve also comprises a valvular structure disposed within frame and configured to regulate the flow of blood through the frame in one direction, at least one an actuation assembly operatively coupled to the frame, and at least one nut coupled to the actuation assembly. The actuation assembly comprises first and second bolts aligned end-to-end relative to each other, wherein an end portion of the first bolt is coupled to an adjacent end portion of the second bolt, wherein the second bolt comprises a threaded portion that extends through and engages internal threads of the nut, and the first and second bolts are rotatable together in a first rotational direction to produce radial expansion of the frame from the radially compressed state to the radially expanded state.

Certain examples concern a prosthetic heart valve, comprising a frame having a first end, a second end, and a longitudinal axis extending between the first end and the second end, wherein the frame is radially expandable between a radially compressed state and a radially expanded state, and wherein the frame comprises a plurality of vertical posts extending parallel to the longitudinal axis and a plurality of angled struts arranged in circumferentially extending rows of angled struts. The prosthetic heart valve also comprises a valvular structure disposed within frame and configured to regulate the flow of blood through the frame in one direction, and at least one actuation assembly comprising a first bolt and a second bolt aligned end-to-end. The first bolt extends through a channel in a first vertical post of the plurality of vertical post, an end portion of the first bolt is coupled to an adjacent end portion of the second bolt, and the first and second bolts are rotatable together in a first rotational direction to produce radial expansion of the frame from the radially compressed state to the radially expanded state. A washer disposed between the adjacent end portion of the second bolt and the first vertical post.

Certain examples concern a prosthetic valve, comprising a radially expandable frame comprising a plurality of interconnected struts, wherein the frame is radially expandable between a radially compressed state and a radially expanded state. The prosthetic valve also includes a valvular structure disposed within frame and configured to regulate the flow of blood through the frame in one direction and at least one an actuation assembly operatively coupled to the frame. The actuation assembly comprises first and second actuation members aligned end-to-end relative to each other, wherein an end portion of the first actuation member is coupled to an adjacent end portion of the second actuation member, wherein the first and second actuation members are rotatable together in a first rotational direction to produce radial expansion of the frame from the radially compressed state to the radially expanded state.

Certain examples concern a prosthetic valve comprising a radially expandable frame, a valvular structure disposed within the frame, and an actuation assembly operatively coupled to the frame. The frame comprises a first end portion, a second end portion, and a plurality of interconnected struts, and is radially expandable between a radially compressed state and a radially expanded state. The valvular structure is configured to permit the flow of blood from the first end portion to the second end portion of the frame and to prevent the flow of blood from the second end portion to the first end portion of the frame. The actuation assembly comprises a stopper having an aperture, an actuation member extending through the aperture in the stopper and a securing pin protruding from at least one side of the actuation member. The securing pin limits motion of the stopper relative to the actuation member in a first axial direction, and the actuation member is rotatable in a first direction to produce radial expansion of the frame from a radially compressed state to a radially expanded state and rotatable in a second rotational direction to produce radial compression of the frame from the radially expanded state to the radially compressed state.

Certain examples concern a prosthetic valve comprising a radially expandable frame, a valvular structure disposed within the frame, and at least one actuation assembly operatively coupled to the frame. The frame comprises a vertically oriented first strut and a vertically oriented second strut, wherein the frame is radially expandable between a radially compressed state and a radially expanded state. The valvular structure is configured to regulate the flow of blood through the frame in one direction. The at least one an actuation assembly comprises an actuation member, a stopper positioned on the actuation member, and a protrusion extending from an outer surface of the actuation member. Bores in the first strut and in the second strut are dimensioned to receive portions of the actuation member. The stopper is positioned between the first strut and the second strut, and the axial movement of the stopper along the actuation member is limited in at least one direction by the protrusion. The actuation member is rotatable in a first direction to move axially with the protrusion contacting the stopper to urge the first strut away from the second strut, thereby radially compressing the frame.

Certain examples concern a medical assembly, comprising a radially expandable frame having a first frame end portion, a second frame end portion, and a longitudinal axis extending between the first end and the second end. The medical assembly also comprises at least one actuation assembly extending longitudinally from the first frame end portion towards the second frame end portion to interconnect first and second portions of the frame. The actuation assembly comprises an actuation member having a first end portion with a head portion adjacent the first frame end and an externally threaded second end portion, a stopper disposed around the actuation member, and a securing pin extending through the actuation member transverse to the longitudinal axis. The securing pin is configured to limit the movement of the stopper along the longitudinal axis in at least one direction. The stopper is configured to limit the movement of the actuation member along the longitudinal axis in at least one direction. The actuation member is rotatable in a first rotational direction to produce radial expansion of the frame from a radially compressed state to a radially expanded state and rotatable in a second rotational direction to produce radial compression of the frame from the radially expanded state to the radially compressed state.

In some examples, a prosthetic heart valve comprises one or more of the components recited in Examples 1-113 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1-2 and an actuator assembly of a delivery apparatus, according to one example.

FIG. 5 is a perspective view of the actuator and actuator assembly of FIG. 4 with the actuator assembly physically coupled to the actuator.

FIG. 6A is a perspective view of a portion of one example of a prosthetic heart valve frame with an actuation mechanism having two bolts.

FIG. 6B is a cutaway side view of the frame segment of FIG. 6A.

FIG. 7 is a schematic illustration of one example actuation assembly having two actuator elements coupled end to end.

FIG. 8 is a schematic illustration of the bolts of the actuator assembly of FIG. 7 further including securing bolts.

FIG. 9A is a perspective view of one example of an actuation stopper.

FIG. 9B is a side elevation view of the actuation stopper of FIG. 9A.

FIG. 9C is a top plan view of the actuation stopper of FIG. 9A.

FIG. 10A is a side elevation view of one example of a frame section and actuation assembly.

FIG. 10B is a perspective view of the frame section and actuation assembly of FIG. 10A.

FIG. 11 is a cutaway top view of the actuation assembly taken along the line 11-11 of FIG. 10A, including actuation member, stopper, and securing pin.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Introduction to the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

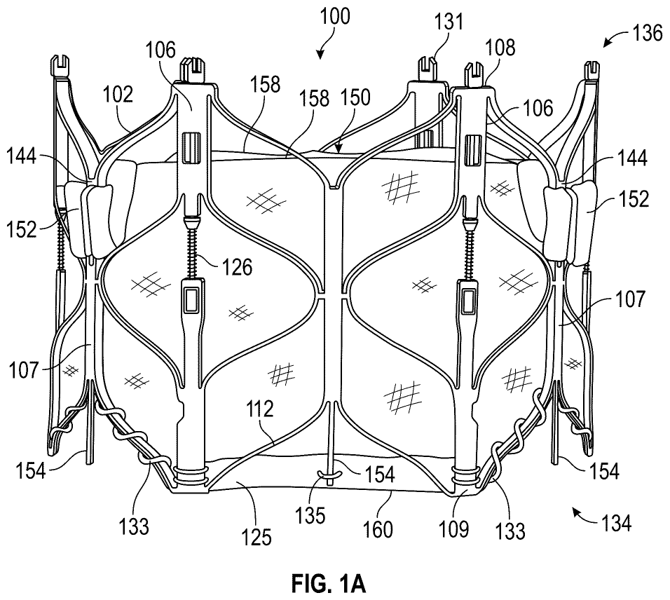

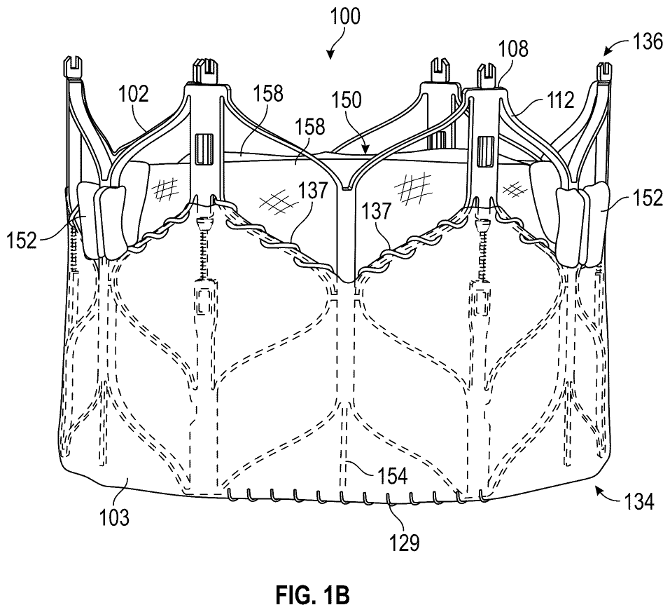

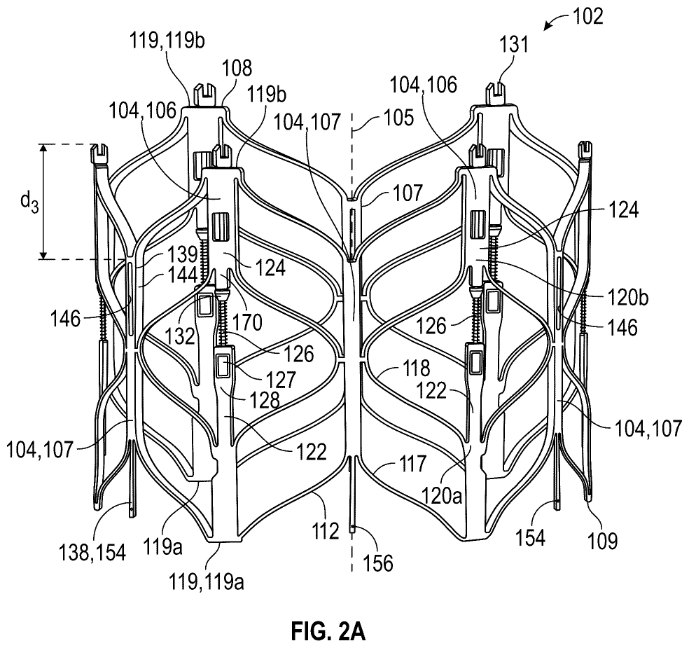

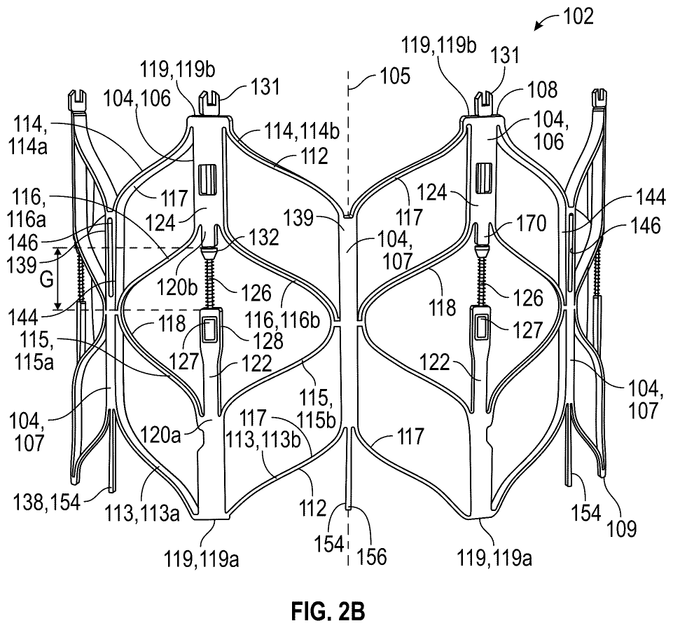

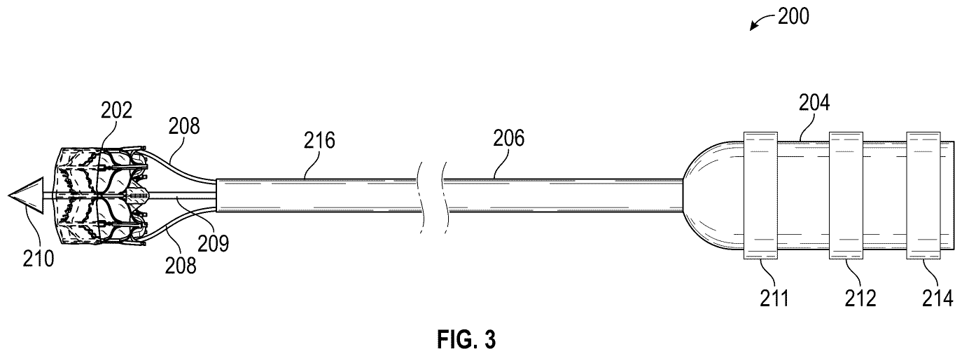

FIGS. 1A-2B illustrate an exemplary prosthetic device (for example, prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

### The Disclosed Technology and Exemplary Embodiments

FIGS. 1A-2B show an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In other examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film including any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In other examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in other examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in other embodiments, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (that is, the lower post shown in FIGS. 2A and 2B) can extend axially from the inflow end 109 of the prosthetic valve 100 toward the second post 124, and the second post 124 (that is, the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore (sometimes referred to as a channel) configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In other examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In other examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In other examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some embodiments, the commissure opening 146 can be fully enclosed by the support post 107 such that a portion of the valvular structure 150 can be slid radially through the commissure opening 146, from an interior to an exterior of the frame 102, during assembly. In the illustrated embodiment, the commissure opening 146 has a substantially rectangular shape that is shaped and sized to receive commissure tabs of two adjacent leaflets therethrough. However, in other embodiments, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d3 (indicated in FIG. 2A). As an example, the offset distance d3 may be in a range from 2 mm to 6 mm. In general, the offset distance d3 should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in other embodiments, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In other examples, the frame 102 can be formed from a self-expandable material (for example, Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As introduced above, the threaded rods 126 can removably couple the prosthetic valve 100 to actuator assemblies of a delivery apparatus. Referring to FIG. 3, it illustrates an exemplary delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated embodiment generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated embodiment shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In other embodiments, a greater or fewer number of actuator assemblies can be present.

In some embodiments, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated embodiment, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated embodiment the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In other embodiments, the first knob 211 can be actuated by sliding or moving the first knob 211 axially, such as pulling and/or pushing the knob. In other embodiments, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In other embodiments, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 202, the delivery apparatus 200 can be removed from the patient, leaving just the prosthetic valve 202 in the patient.

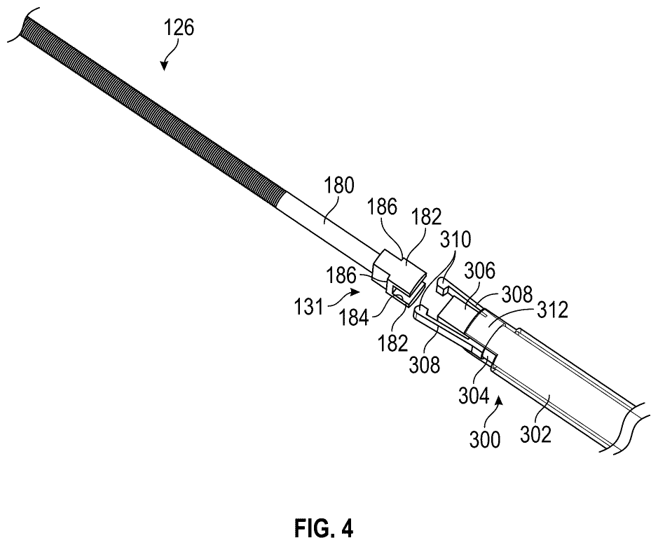

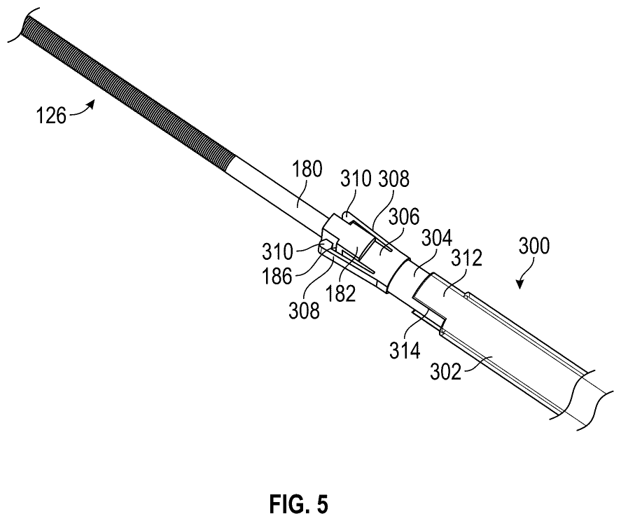

Referring to FIGS. 4-5, they illustrate how each of the threaded rods 126 of the prosthetic device 100 can be removably coupled to an exemplary actuator assembly 300 (for example, actuator assemblies 208) of a delivery apparatus (for example, delivery apparatus 200). Specifically, FIG. 5 illustrates how one of the threaded rods 126 can be coupled to an actuator assembly 300, while FIG. 4 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this embodiment can include the same features described previously for delivery apparatus 200. In particular embodiments, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In other embodiments, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (that is, over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated embodiment the central protrusion 306 has a substantially rectangular shape in cross-section, in other embodiments, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

Also disclosed herein are examples of actuation assemblies having two bolts or rods coupled end-to-end. In some prosthetic heart valves, the portions of the actuation assembly closer to the distal and/or proximal ends of the frame may experience less compressive and/or flexural stress than portions of the actuation assembly closer to the axial midpoint of the prosthetic heart valve frame. If the actuation assembly does not have sufficient strength to withstand these compressive and/or flexural stresses, parts of the actuation assembly could buckle, particularly when the prosthetic heart valves are retained in the compressed configuration. Accordingly, those parts of the actuation assembly nearer to the axial midpoint of the frame require greater thickness (and thus greater strength) than the parts of the actuation assembly nearer the ends of the frame. However, in actuation assemblies comprising only one bolt or otherwise comprising only a single, unitary actuator body, the entire length of the actuator must be of sufficient thickness to withstand the compressive and/or flexural forces at the highest point of stress along the axial length of the actuation assembly. At the same time, thinner actuation assemblies are desirable because the thickness of the actuation assembly controls the minimum thickness of any components of the frame through which those assemblies extend. A frame with thinner components can be radially compressed to a smaller diameter in the fully compressed state, allowing for greater ease of delivery and implantation. Accordingly, the design of actuation assemblies for prosthetic heart valves faces two competing demands: the desire for minimal component thickness, and the need to withstand compressive and/or flexural stresses on the actuation assembly components.

This may be addressed through the use of actuation assemblies comprising two connected members, such as in the form of bolts, arranged end to end, such as those discussed in greater detail below, allowing for a bolt positioned nearer to the axial midpoint of the frame to be designed with a greater thickness than a bolt positioned towards one end of the frame. Additionally, this allows the use of thinner bolts in the portions of actuation assemblies positioned nearer to one end of the frame, as these bolts need only be strong enough to withstand the lesser compressive and/or flexural stresses experienced near the ends of the frame, and to rotate the bolts positioned nearer to the axial midpoint of the frame. The use of thinner bolts in the portions of the actuation assemblies positioned nearer to a frame end in turn allows for a thinner overall frame profile, while the use of thicker, stronger bolts in portions of the actuation assemblies positioned nearer to the axial midpoint of the frame allows for actuator assembly designs capable of withstanding greater compressive and/or flexural stresses when the frame (and thus the prosthetic heart valve) is in the compressed configuration.

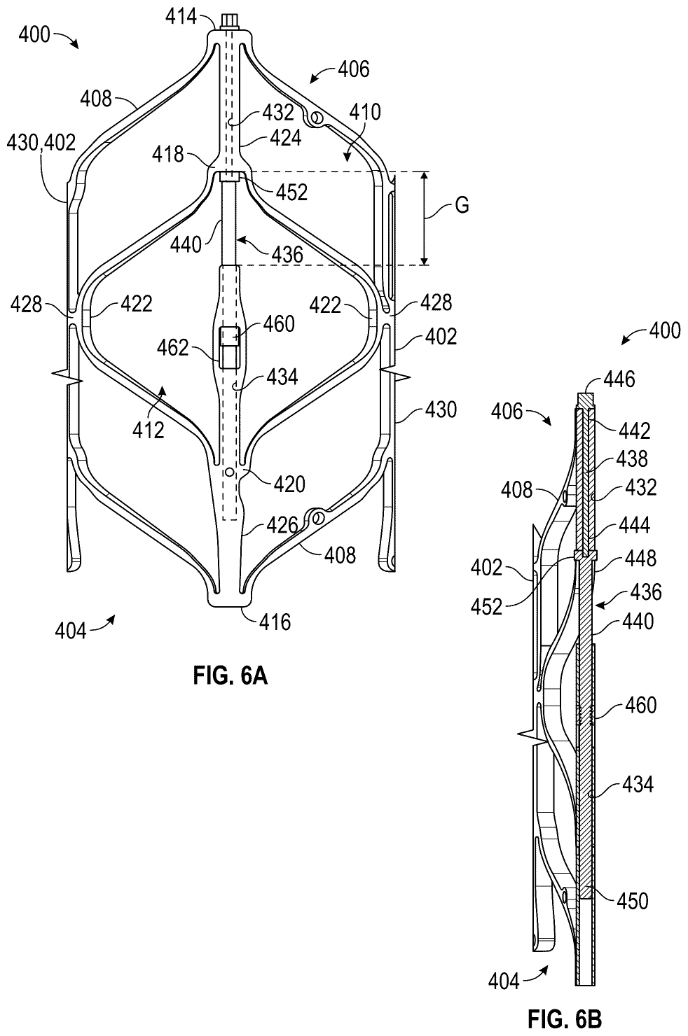

FIGS. 6A and 6B show a section of an exemplary prosthetic heart valve frame 400 having an actuator comprising two separate members in the form of bolts. The frame 400 can comprise a plurality of vertically oriented posts 402 extending from an inflow end 404 towards an outflow end 406 of the frame 400, and connected by a plurality of circumferentially extending link members or struts 408. Each strut 408 extends between adjacent posts 402 to connect all of the vertically oriented posts 402. A plurality of frame sections such as that shown in FIG. 6A can be arranged side by side, such that each frame section is adjacent to two neighboring frame sections, and adjacent frame sections share a vertically oriented post 402, in an annular pattern to form the frame 400.

As illustrated in FIG. 6A, the axially extending posts 402 and circumferentially extending struts 408 can form and/or define a plurality of cells (that is, openings) of the frame 400. For example, the posts 402 and the struts 408 can form or define a plurality of first cells (sometimes called outer cells) 410 and a plurality of second cells (sometimes called inner cells) 412 that extend circumferentially around the frame 400. Specifically, each first cell 410 can be formed by two axially extending posts 402 and four circumferentially extending struts 408, and each second cell 412 can be formed by four circumferentially extending struts 408.

Each first cell 410 can have a hexagonal shape, including a first apex (sometimes called an outflow apex) 414 positioned at the outflow end 404 of the frame 400 and a second apex (sometimes called an inflow apex) 416 positioned at the inflow end 404 of the frame 400. In examples where the delivery apparatus is releasably connected to the outflow apices 414, as previously described, each inflow apex 416 can be referred to as a “distal apex” and each outflow apex 412 can be referred to as a “proximal apex”. In other examples where the delivery apparatus is releasably connected to the inflow apices, each inflow apex 416 may be referred to as a “proximal apex” and each outflow apex 412 may be referred to as a “distal apex” While FIG. 6A shows the first cell 410 having a hexagonal shape, formed by two posts 402 and four struts 408, it is to be understood that that in other examples, the first cell 410 can be formed from a differing number of struts and/or posts and have a different geometry.

Each second cell 412 can be formed by four circumferentially extending struts 408 and can be disposed within a corresponding first cell 410 as shown in FIG. 6A. Each second cell 412 can have a diamond shape including a first apex (sometimes called an outflow apex) 418 and a second apex (sometimes called an inflow apex) 420 and two lateral vertices 422. As shown in FIG. 6A, the second cell 412 may be connected to a corresponding first cell 410 by a first post 424 extending from the first apex 414 of the first cell 410 to the first apex 418 of the second cell 412, and by a second post 426 extending from the second apex 420 of the second cell 412 to the second apex 416 of the first cell 410. The second cell 412 may also be connected to the corresponding first cell 410 by lateral members 428 extending from the lateral vertices 422 to the nearest adjacent axially oriented post 402 of the corresponding first cell 410, as shown in FIG. 6A. It is to be understood that while FIG. 6A shows a second cell 412 formed by four circumferentially extending struts 408 and having a diamond shape, in other examples, the second cell 412 can be formed from a different combination of struts 408 and/or posts 402, and may have a different geometry.

In some examples, the frame 400 comprises six first cells 410 extending circumferentially in a row, six second cells 412 extending circumferentially in a row within the six first cells 410, and twelve posts 402. However, in other embodiments, the frame 400 can comprise a greater or fewer number of first cells 410 and a correspondingly greater or fewer number of second cells 412 and posts 402.

Although only one first cell 410 and one second cell 412 is shown in FIG. 6A, it should be understood that a complete frame 400 include multiple pairs of such cells 410, 412 connected side-by-side, in the same manner as the frame 102 of FIGS. 2A-2B. Moreover, a prosthetic valve can comprise the frame 400 and the other components of the prosthetic valve 100 of FIGS. 1A-1B, including leaflets 158 and a skirt 103.

In some examples, the first posts 424 and the second posts 426 can be arranged in pairs of axially aligned vertical posts along the length of the frame 400. The first and second posts 424, 426 axially separated from one another by a gap G. Each first post 424 (that is, the upper post shown in FIG. 6A) can extend axially from the outflow end 406 of the frame 400 toward the second post 426, and the second post 426 (that is, the lower post shown in FIG. 6A) can extend axially from the inflow end 404 of the frame 400 toward the first post 424. In other words, each first post 424 can be connected to and extend from an outflow apex 414 and each second post 426 can be connected to and extend from an inflow apex 416.

Other posts 402 can be support posts 430, extending from the inflow end 404 and the outflow end 406 of the frame 400. In one example, the frame 400 can comprise an equal number of support pots 430 and pairs of axially aligned posts 424, 426 arranged in an alternating order such that each circumferentially extending strut 408 extends from one of the pairs of axially aligned posts 424, 426 to one of the support posts 430 (that is, each strut 408 can be coupled on one end to one of the posts 424, 426 and can be coupled on the other end to one of the support posts 430). However, it is to be appreciated that in other examples, the frame 400 can have differing numbers of support posts 430 and pairs of axially aligned posts 424, 426, or that the support posts 430 and pairs of axially aligned posts 424, 426 may be arranged in a non-alternating order.

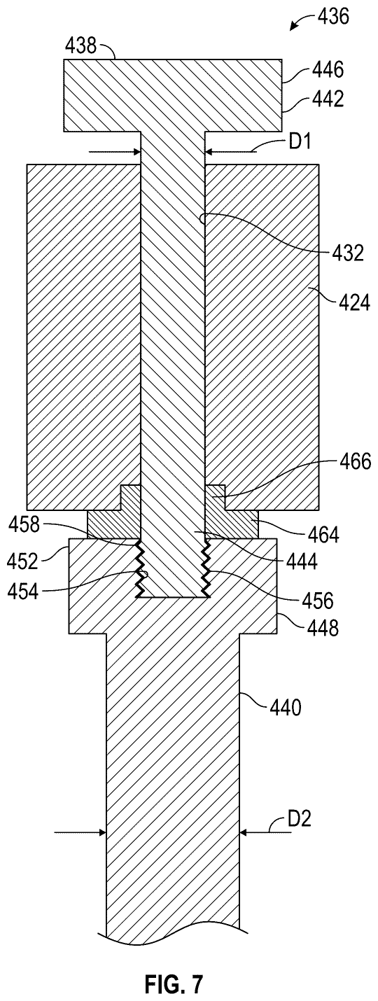

In some examples, as illustrated in FIGS. 6A and 6B, the first post 424 of each pair of axially aligned vertical posts can include a first internal axially extending bore 432 (sometimes referred to as a channel 432) and the second post 426 can include a second internal axially extending bore 434. The first and second internal bores 432, 434 can be configured to receive portions of an actuation assembly, such as the threaded rods (or bolts) of actuation assembly 436 as shown in FIGS. 6A and 6B, and illustrated in greater detail in FIG. 7. In some examples, an actuation assembly 436 can extend through each pair of axially aligned posts 424, 426 so that all of the posts 424, 426 (with their corresponding actuation mechanism 436) serve as expansion and locking mechanisms for the frame 400. As just one example, the frame 400 can include six pairs of posts 424, 426, and each of the six pairs of posts 424, 426 with their corresponding actuation assemblies for a total of six expansion and locking mechanisms. In other examples, not all pairs of posts 424, 426 need be expansion and locking mechanisms (that is, actuators). If a pair of posts 424, 426 is not used as an expansion and locking mechanism, an actuation assembly 436 need not extend through the posts 424, 426 of that pair.

Turning now to FIG. 6B, the actuation assembly 436 can include a first actuation member in the form of a first bolt 438 and a second actuation member in the form of a second bolt 440. The first bolt 438 can have a first end portion 442 and a second end portion 444 and can extend through the first internal bore 432. In examples where the frame 400 is configured to releasably attach to a delivery apparatus at the outflow end 406, the first end portion 442 of the first bolt 438 can be referred to as the proximal end portion of the first bolt 438 and the second end portion 444 of the first bolt 438 can be referred to as the distal end portion of the first bolt 438. The first bolt 438 can extend proximally past the proximal end of the first post 424 and the first end portion 442 can include an enlarged head portion 446 at its proximal end that can serve at least two functions. First, the head portion 446 can removably or releasably couple the first bolt 438 to a respective actuator assembly of a delivery apparatus that can be used to radially expand and/or radially compress the frame 400, as previously discussed. Second, the head portion 446 can prevent the first post 424 from moving proximally relative to the first bolt 438 and can apply a distally directed force to the first post 424, such as when radially expanding the frame 400, as previously discussed.

The second bolt 440 can have a first end portion 448 and a second end portion 450, and can extend through the second internal bore 434. As illustrated in FIGS. 6A and 6B, the first end portion 448 of the second bolt 440 can include an enlarged head portion 452. In some examples, the head portion 452 of the second bolt 440 serves the same function as the stopper 132 in the example of FIGS. 1-2. Thus, when the actuation assembly 436 is rotated to radially compress the frame 400, the enlarged head portion 452 can apply a proximally directed force to the first post 424 to move the first post 424 away from the second post 426, thereby increasing gap G and radially compressing the frame. Use of a separate stopper that is welded to an actuator member can weaken the actuation assembly and increase the complexity of the manufacturing process. Advantageously, the enlarged head portion 452 is integrally formed as part of the second bolt 440 and does not need to be separately formed and welded to the second bolt. Thus, use of the integral head portion 452 as the stopper can overcome the deficiencies of a separately formed stopper.

The first bolt and the second bolt may be coupled to each other end-to-end, as best shown in FIG. 7. For example, the second end portion (for example, the distal end portion) 444 of the first bolt 438 can be directly attached to the first end portion (for example, the proximal end portion) 448, particularly the head portion 452, of the second bolt 440. In some examples, the head portion 452 of the second bolt 440 can include a bore 454 that receives a portion of the second end portion 444 of the first bolt 438. In some examples, as illustrated in FIG. 7, the bore 454 can include internal threads 456, which may threadably engage corresponding external threads 458 on the second end portion 444 of the first bolt 438.

It is to be appreciated, however, that in other examples, other methods and/or techniques may be used to attach the first bolt 438 to the second bolt 440. For example, in lieu of or in addition to the threaded connected between the first and second bolts, the actuation assembly 436 can include one or more pins extending through both the second end portion 444 of the first bolt 438 and the first end portion 446 of the second bolt 440, or any other device or method suitable for coupling the first bolt 438 and the second bolt 440.

For example, as illustrated in FIG. 8, the first bolt 438 and the second bolt 440 can be coupled by a first pin 468 extending through both the first bolt 438 and the second bolt 440. In some examples, the actuation assembly 436 can also include a second pin 470 extending through both the first bolt 438 and the second bolt 440. As shown, the pins 468, 470 can extend through the head portion 452 of the second bolt 440 and the end portion 444 of the first bolt 438. It should be understood that the threaded connection between the bolts shown in FIG. 8 is optional and can be removed. While FIG. 8 shows that the first pin 468 and the second pin 470 can be perpendicular or substantially perpendicular to each other, it is to be appreciated that in some examples, the first pin 468 and the second pin 470 can have a different orientation relative to each other.

In another example, the position of the external and internal threads can be reversed. Specially, the end portion 448 of the second bolt 440 can be formed with external threads and the end portion 444 of the first bolt 438 can be formed with a bore having internal threads, such that the end portion 448 can be tightened into the bore of the end portion 444.

In these examples, the first bolt 438 and the second bolt 440 are rotationally fixed to each other, such that rotating the first bolt 438 in a first rotational direction (for example, when the first bolt is rotated in a first rotational direction by a component of a delivery system such as actuator 300), also rotates the second bolt 440 in the first rotational direction.

In other examples, the first and second bolts 438, 440 can be indirectly coupled to each other by one or more intermediate components. For example, a coupling member can be disposed between and couple the adjacent end portions of the first and second bolts 438, 440 to each other. For example, the coupling member can comprise a sleeve coupling having internal threads that engage external threads of the first and second bolts. Thus, the second end portion 444 of the first bolt 438 having external threads 458 can be tightened into the sleeve coupling. Likewise, the first end portion 448 of the second bolt 440 can have external threads such that the first end portion 448 can be tightened into the sleeve coupling.

In further examples, the actuation assembly 436 can include more than two actuation members (for example, bolts), such as three or more actuation members (for example, bolts), coupled end-to-end to each other using any of the coupling techniques and/or mechanisms described above for the first and second bolts 438, 440.

In some examples, best shown in FIGS. 6A and 6B, the second bolt 440 can also extend through a bore of a nut 460 captured within a window (sometimes called a slot) 462 formed in the second post 426. In such examples, the nut 460 may have internal threads that engage with corresponding external threads of the second bolt 440. Rotation of the second bolt 440 relative to the nut 460 produces radial expansion and compression of the frame 400, as further described below. In such examples, interior of the second bore 434 (through which the second bolt 440 extends) can have a smooth and/or non-threaded inner surface to allow the second bolt 440 to slide freely within the bore. It is to be appreciated, however, that in other examples, the actuation assembly 436 can omit the nut 460, and in such examples the interior of the second bore 434 may include an internal thread that engages with corresponding external threads on the second bolt 440.

In some examples, the second bolt 440 can extend past the nut 460 toward the inflow end 404 of the frame 400 and into the second bore 434. The nut 460 can be held in a fixed position relative to the second post 426 such that the nut 460 does not rotate relative to the second post 426. In this way, whenever the second bolt 440 is rotated (for example, when the first bolt 438 is rotated) the threaded the second bolt 440 can rotate relative to both the nut 460 and the second post 426. The engagement of the external threads of the second bolt 440 and the internal threads of the nut 460 prevent the second bolt 440 from moving axially relative to the nut 460 and the second post 426 unless the second bolt 440 is rotated relative to the nut 460. Thus, the second bolt 440 can be retained or held by the nut 460 and can only be moved relative to the nut 460 and/or the second post 426 by rotating the second bolt 440 relative to the nut 460 and/or the second bolt 440. In other examples, in which a portion of the second bore 434 is threaded in lieu of using the nut 460, the rotation of the second bolt 440 causes the second bolt 440 to move axially relative to the second post 426.

The actuation assembly 436 can be rotated relative to the nut 460, the first post 424, and the second post 424 to axially foreshorten and/or axially elongate the frame 400, thereby radially expanding and/or radially compressing, respectively, the frame 400 (and therefore any prosthetic heart valve including the frame 400). Specifically, when the first bolt 438 is rotated relative to the first post 424, the second bolt 440 rotates along with the first bolt 438 relative to the nut 460 and the second post 426. The first post 424 and the second post 426 can move axially relative to one another, by deflecting or bending struts 408 under the axial force applied by the actuation assembly 436, thereby widening or narrowing the gap G (FIG. 6A) separating the posts 424, 426. This thereby radially compresses or radially expands the frame 400 respectively. Thus, the gap G (FIG. 2B) between the first and second posts 424, 426 narrows as the frame 400 is radially expanded and widens as the frame 400 is radially compressed.

Rotation of the first bolt 438 and the second bolt 440 of the actuation assembly 436 in a first rotational direction (for example, clockwise) can cause corresponding axial movement of the first and second posts 424, 426 toward one another, thereby decreasing the gap G and radially expanding the frame 400, while rotation of the bolts 438 and 440 in an opposite second rotational direction causes corresponding axial movement of the first and second posts 424, 426 away from one another, thereby increasing the gap G and radially compressing the frame. When first bolt 438 is rotated in the first direction, the head portion 446 of the first bolt 438 bears against an adjacent surface of the frame (for example, an outflow apex 414), while the nut 127 and the second post 426 travel proximally along the second bolt 440 toward the first post 424, thereby radially expanding the frame. As the frame 400 moves from a compressed configuration to an expanded configuration, the gap G between the first and second posts 424, 426 can narrow.

When the first bolt 438 is rotated in the second direction, the second bolt 440 moves toward the outflow end 406 of the frame 400 until the head portion 452 abuts the distal end of the first post 424 or an optional washer 464 coupled to the distal end of the first post (as shown in FIG. 7). Upon further rotation of the first bolt 438 in the second direction, the head portion 452 can apply a proximally directed force to the first post 424 to radially compress the frame 400. Specifically, during crimping/radial compression of the prosthetic valve frame 400, the first bolt 438 can be rotated in the second direction (for example, counterclockwise) causing the head portion 452 to push against (that is, provide a proximally directed force to) the distal end of the first post 424 (or against the washer 464 coupled to the first post 424), thereby causing the first post 424 to move away from the second post 426, and thereby axially elongating and radially compressing the frame 400.

Thus, each of the first posts 424 can slide axially relative to a corresponding one of the second posts 426 but can be axially retained and/or restrained between the head portion 446 of the first bolt 438 and the head portion 442 of the second bolt 440. That is, each first post 424 can be restrained at its proximal end by the head portion 446 of the first bolt 438 and at its distal end by the head portion 452 of the second bolt. In this way, the head portion 446 of the first bolt 438 can apply a distally directed force to the first post 424 to radially expand the frame 400 while the head portion 452 can apply a proximally directed force to the first post 424 to radially compress the frame 400. As explained above, radially expanding the frame 400 axially foreshortens the frame 400, causing the inflow end 404 and the outflow end 406 (FIGS. 6A and 6B) to move towards one another axially, while radially compressing the frame 400 axially elongates the frame 400, causing the inflow end 404 and outflow end 406 of the frame 400 to move away from one another axially.

In other examples, the first bolt 438 can be fixed against axial movement relative to the first post 424 such that rotation of the first bolt 438 in the first direction produces rotation of the second bolt 440 in the first direction, proximal movement of the nut 460, and radial expansion of the frame 400 and rotation of the first bolt 438 in the second direction produces rotation of the second bolt 440 in the second direction, distal movement of the nut 460 and radial compression of the frame 400.

In some examples, as shown in FIG. 7, the actuation assembly 436 also can include an optional washer 464. The washer 464 can be coupled to the distal end of the first post 424 and can have a central aperture aligned with the bore 432 such that the distal end portion 444 of the first bolt 438 can extend through the washer. In some examples, such as that illustrated in FIG. 7, the washer 464 is positioned between the head portion 452 of the second bolt 440 and the distal end of the first post 424 to reduce wear between these two components. The washer 464 can also include a sleeve portion 466 that extends axially into the first internal bore 432 in the first post 424, as illustrated in FIG. 7. The washer 464 can be fixed to the first post 424, such as with an adhesive, welding, and/or via a press fit between the sleeve portion 466 and the inner surface of the bore 432, and/or other techniques or mechanisms. Advantageously, the sleeve portion 466 may seal the first internal bore 432, preventing the formation of thromboses or the occurrence of crevice corrosion after a prosthetic heart valve including frame 400 has been implanted in the vasculature of a patient. Although not shown, another washer 464 can be positioned between the head portion 446 of the first bolt 438 and the proximal end of the first post 424. The additional washer 464 can include a sleeve portion 466 that extends into the bore 432 at the proximal end of the first post 424 to seal that end of the first post.

Because the actuation assembly 436 includes two separate bolts, each bolt 438, 440 can be easily manufactured to have different dimensions and/or shapes, and/or can be manufactured from different materials to optimize the performance of the frame. For example, as noted above, when radially compressed, the frame experiences the greatest compressive and/or flexural stresses at the along the middle of the frame. Thus, as shown in FIG. 7, the first bolt 438 can have a first diameter D1, and the second bolt 440 can have a second diameter D2. The first diameter D1 can, as illustrated in FIG. 7, be less than the second diameter D2. In one particular example, the first diameter D1 can be 0.55 mm, and the second diameter can be 0.78 mm. In other examples, the first diameter D1 can be greater or less than 0.55 mm, such as between 0.35 and 0.75 mm, or 0.35 mm, 0.40 mm, 0.45 mm, 0.50 mm, 0.55 mm, 0.60 mm, 0.65 mm, 0.75 mm, or any diameter in between. In other examples, the second diameter D2 can be greater or less than 0.78 mm, such as 0.55 mm to 0.95 mm, or 0.55 mm, 0.60 mm, 0.65 mm, 0.70 mm, 0.75 mm, 0.80 mm, 0.85 mm, 0.90 mm, 0.95 mm, or any diameter in between.

By forming the second bolt 440 with a greater diameter, it can better resist buckling when the frame is radially compressed. Further, due to the increased strength provided by the relatively wider second bolt 440, the overall length of the second post 426 (compared to bolt 126 in the example of FIGS. 2A-2B) can be reduced, which reduces the amount of metal along the middle portion of the frame and therefore reduces the crimp profile along this portion of the frame. Because the compressive and/or flexural stresses along the outflow end portion of the frame are relatively smaller than the middle portion of the frame, the diameter of the first bolt 438 can be relatively smaller than the second bolt 440. This allows the first post 424 to have a relatively smaller thickness and/or width compared to the second post 426, which reduces the overall frame thickness and crimp profile the frame along the outflow end portion when in the radially compressed configuration.

In lieu or in addition to utilizing bolts with different diameters, the first and second bolts can be formed from different materials. For example, the second bolt 440 can be formed a relatively stiffer or stronger material than the material used to form the first bolt 438 to better resist the compressive and/or flexural forces along the middle portion of the frame when the frame is radially compressed.

Also disclosed herein are examples of actuation assemblies having a stopper (sometimes in the form of a washer) secured by a pin extending laterally through the actuation member. As discussed above, actuation assemblies for prosthetic heart valves may include a stopper to facilitate the radial expansion and/or compression of the frame. To enable the radial expansion and compression of the frame, the stopper is fixed relative to the actuation member, such that as the actuation member moves axially relative to the frame, the stopper may apply axial forces to the components of the frame. These stoppers may, in some examples, be welded to the actuation member or secured by mechanical deformation such as crimping or stamping. When the stopper is attached to the actuation member in these ways, however, the actuator and/or the stopper may be mechanically weakened. When the stopper is attached by welding, the localized melting and re-solidification of the material of the actuation member can cause localized embrittlement or loss of strength, and portions of the actuation member adjacent to the weld site may be weakened from residual heat from the welding process. Additionally, welded sections of material may be particularly susceptible to corrosion after re-solidification. When the stopper is attached by mechanical deformation such as stamping or crimping, the actuator and/or stopper may become deformed or damaged, may lose mechanical strength, and/or may experience reduced fatigue tolerance.

This may be addressed through the use of actuation assemblies having a securing pin to fix the stopper relative to the actuation member. The securing pin can extend laterally through the actuation member and can be sized such that the stopper cannot travel axially along the actuation member past the position of the securing pin. In this way, the need to physically attach the stopper to the actuation member by welding or mechanical deformation can be avoided. Additionally, because the stopper does not need to be welded or mechanically deformed onto the actuation member, the stopper can be formed of a wider range of materials, such as polymer materials or metals which do not readily weld to the materials used for the actuation member. Advantageously, the use of polymer materials may also reduce the friction between the frame, the actuator, and the stopper, which can reduce resistance to the rotation of the actuators during the radial expansion and/or radial compression of the frame.

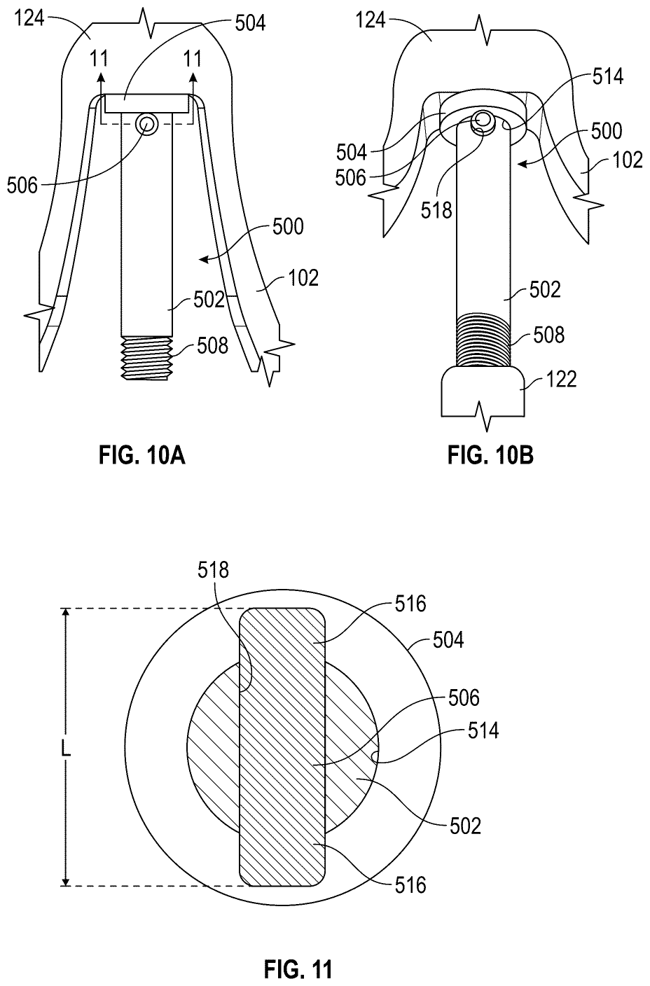

FIGS. 10A and 10B show a portion of an actuation assembly 500 comprising a securing pin 506 to restrain a stopper 504. The actuation assembly 500 can be operatively attached to a frame similar to the frame 102, described in greater detail above, having first posts 122 and second posts 124 axially aligned with each other to form pairs of posts. The actuation assembly 500 generally comprises an actuation member 502, the stopper 504, and the securing pin 506. As illustrated, the actuation member 502 extends axially between one of the first posts 122 and an aligned one of the second posts 124, and through one or more axially aligned bores (see, for example, FIGS. 6A and 6B). In some examples, the actuation member 502 can have an externally threaded end portion 508 to engage with other components of the frame and/or the actuation assembly, such as the nut 127 (see, for example, FIG. 2A), as discussed in greater detail above.

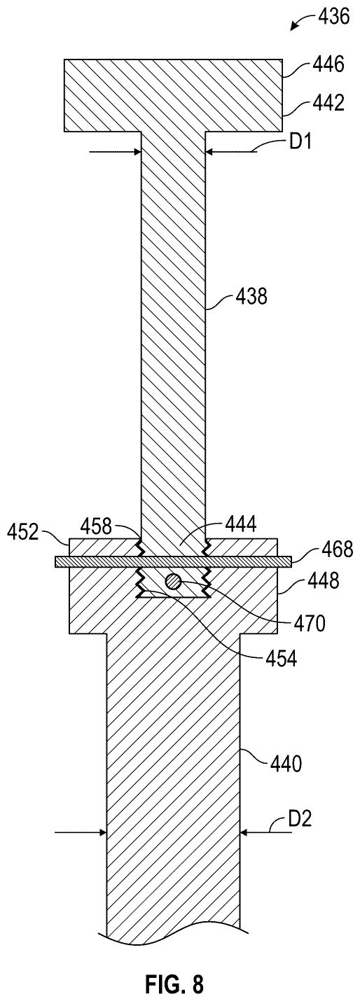

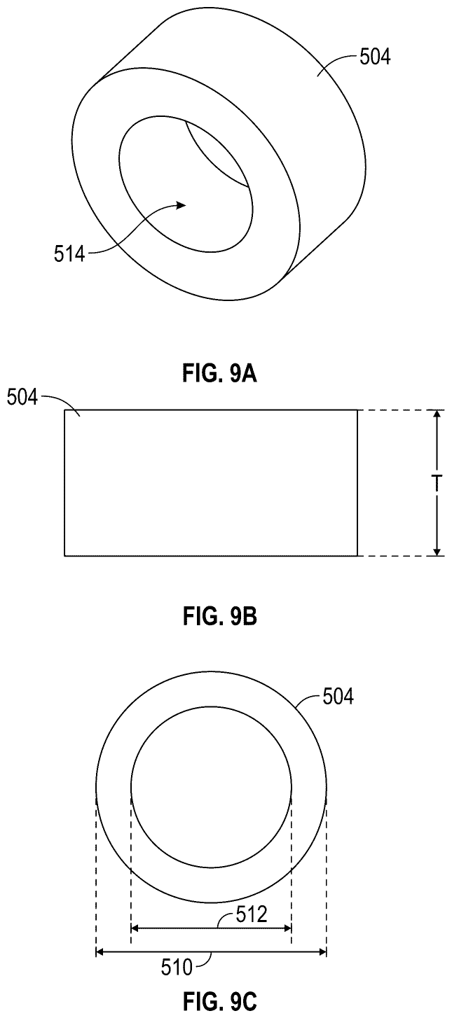

As also illustrated in FIGS. 9A through 9C, the stopper 504 can comprise an annular body with an outer diameter 510 and an aperture 514 having an inner diameter 512. The aperture 514 receives the actuation member 502 (that is, the stopper 504 is disposed radially around the actuation member 502). The dimensions of the outer diameter 510 and the inner diameter 512 may be defined by the dimensions of the other components of the frame 102. For instance, in some examples, the outer diameter 510 may be sized such that the stopper 504 does not exceed the thickness of the first post 122 and/or the second post 124, so as not to increase the crimp profile of the frame 102 in a radially compressed configuration. In some specific examples, the outer diameter 510 may be less than or equal to 1 mm, such as 0.9 mm or less, 0.8 mm or less, 0.7 mm or less, or 0.6 mm or less. In other examples, the outer diameter 510 may be greater than 1.0 mm. The inner diameter 512 may similarly be defined by the outer diameter of the actuation member 502, such that the inner diameter 512 exceeds the outer diameter of the actuation member 502 only by a small margin so that the stopper 504 can slidingly translate relative to the actuation member 502.

The stopper 504 also has a thickness T, as best illustrated in FIG. 9B. The thickness T of the stopper 504 is generally sufficiently large that the stopper 504 has adequate mechanical strength to withstand the forces associated with radially expanding and compressing the frame, but also not so great that the stopper 504 prevents the first post 122 and the second post 124 from achieving the spacing required to radially expand the frame to its maximum desired diameter. For some example frames, the thickness T of the stopper 504 is 0.5 mm or less. For other example frames, the thickness T of the stopper can be greater than 0.5 mm.

The stopper 504 can be formed of any suitable biocompatible material with sufficient mechanical strength to withstand the compressive and other forces that the stopper 504 experiences during the radial compression and radial expansion of the frame 102. Such materials include biocompatible alloys including cobalt-chromium alloys, and biocompatible polymers such as polyether ether ketone, teflon, polyethylenimine, or a combination thereof.

The dimensions (such as the outer diameter 510, the inner diameter 512, and the thickness T) of the stopper 504 can be varied for frames with a variety of configurations and dimensions and selected such that the stopper 504 is configured to withstand a given compressive force. In some examples, the given compressive force can be at least 20 N, such as 20 N, 30 N, 40 N, 50 N, 60 N, 70 N, or 80 N. In one specific working example, the stopper 504 can be formed from a cobalt-chromium alloy, with a thickness T of about 0.4 mm, an outer diameter 510 of about 0.8 mm, and an inner diameter 512 of about 0.56 mm, and can be configured to withstand a compressive force of at least 40 N.

Turning now to FIG. 11, the securing pin 506 can extend laterally through the cross section of the actuation member 502, such that at least one end portion 516 protrudes beyond an outer surface of the actuation member 502 (see for example, FIG. 10B). A length L of the securing pin 506 may be varied depending on the geometries of the actuation member 502 and the stopper 504. In the illustrated example, the length L is greater than the outer diameter of the actuation member 502, such that both of the end portions 516 can extend radially outwards from the actuation member 502. Similarly, the length L is generally less than the outer diameter 510 of the stopper 504, so that the securing pin 506 does not increase the crimp profile of the frame 102.

In some examples, the securing pin 506 can be integrally formed with the actuation member 502. In other examples, the actuation member 502 can comprise a slot or aperture 518 configured to accommodate the securing pin 506. In such examples, the securing pin 506 can be press fit into the slot or aperture 518, or thermally fitted into the slot or aperture 518. While FIGS. 10A and 10B show a securing pin 506 having a circular cross section, the securing pin 506 may have other cross-sectional geometries, such as a rectangular cross section, an irregularly-shaped cross section, or any other cross section that allows the securing pin 506 to be fixed relative to the actuation member 502, and to secure the stopper 504.

The cross-sectional area of the securing pin 506 may be determined by the mechanical demands on both the stopper 504 and the actuation member 502. The cross-sectional area of the securing pin 506 should be great enough to withstand the axial forces applied to the stopper 504 during the radial compression (that is, the axial expansion) of the frame 102. If the cross-sectional area of the securing pin 506 is too small, it may shear or buckle during the radial compression of the frame 102. However, if the cross-sectional area of the securing pin 506 is increased, then the size of any slot or aperture in the frame to accommodate the securing pin 506 is also increased, which in turn may reduce the mechanical strength of the actuation member 502, which should remain sufficient to withstand tensile and/or rotational stresses associated with the radial expansion and/or compression of the frame 102.

The maximum and minimum diameter of the securing pin 506 can be varied for frames with a variety of configurations and dimensions and selected such that the pin 506 can withstand a given compressive force, and such that the actuation member 502 with the aperture 518 extending therethrough can withstand a given tensile stress. In some examples, the given compressive force can be at least 20 N, such as 20 N, 30 N, 40 N, 50 N, 60 N, 70 N, or 80 N, and the given tensile stress can be at least 200 MPa, such as 200 MPa, 300 MPa, 400 MPa, 500 MPa, 600 MPa, 700 MPa, or 800 MPa. In one specific working example, the actuation member 502 with a diameter of approximately 0.55 mm and the securing pin 506 extending through the actuation member has a diameter of 0.25 mm. The securing pin 506 is configured to withstand an axial force of at least 40 N, and the actuation member 502 is configured to withstand tensile stresses of at least 400 MPa.

Returning now to FIGS. 10A and 10B, the securing pin 506 can be positioned axially between the first post 122 and the second post 124. As also shown in FIGS. 10A and 10B, the stopper 504 in some examples can be positioned between the securing pin 506 and the second post 124, and the securing pin 506 can be positioned between the stopper 504 and the first post 122. In such examples, the stopper 504 can be bounded on one side by an end portion of the second post 124 and on the other side by the securing pin 506.

The actuation assembly 500 can operate similar to the threaded rod 126 and the stopper 132 described in greater detail above, except that the stopper 504 is not integrally formed with or fixedly attached to the actuation member 502, and thus does not necessarily move in lockstep or rotate with the actuation member 502. Rather, the securing pin 506 is integrally formed with or fixedly attached to the actuation member 502 and provides a limit on the axial movement of the stopper 504 relative to the actuation member 502 in one direction. The axial movement of the stopper 504 relative to the actuation member 502 in an opposite direction can be limited by the second post 124.

Because the stopper 504 is not integrally formed with or fixedly attached to the actuation member 502, the movement of the stopper relative to the frame 102 can be reduced or eliminated. In some examples, when the actuation member 502 is rotated, the actuation member 502 can rotate relative to the stopper 504 such that an outer surface of the actuation member 502 rotates along the inner diameter 512 of the aperture 514, and the securing pin 506 can rotate along with the actuation member 502 and travel along an axial surface of the stopper 504. In some examples, the stopper 504 may remain stationary relative to the frame 102 as the frame is radially expanded and/or radially compressed. In other examples, the stopper 504 may rotate relative to the frame 102 to a lesser degree than the rotation of the actuation member 502 and the securing pin 506 relative to the frame 102. This in turn can reduce the resistance to the rotation of the actuation member 502 imparted by the stopper 504 and can also reduce the wear occurring from friction between components of the actuation assembly 500 and the frame 102.

In the illustrated example, for example, when the actuation member 502 is rotated to radially compress the frame, the actuation member 502 and securing pin 506 translate towards the second post 124. As the securing pin 506 travels towards the second post 124, it urges the stopper 504 along with it until the stopper 504 abuts an end portion of the second post 124. Further rotation of the actuation member 502 in the same direction causes the stopper 504 to bear on the end portion of the second post 124, applying a proximally directed force to the second post 124 and pulling the second post 124 axially away from the first post 122 and thereby axially compressing the frame 102. In the illustrated example, the actuation member 502 is threadedly engaged such that its rotation causes axial movement. The stopper 504 is slidingly engaged with the actuation member 502, and thus it may or may not rotate as the actuation member 502 is threaded or unthreaded.

Radially expanding and radially compressing the frame can be accomplished as previously described for the examples of FIGS. 1-8. Thus, the actuation member 502 can include an enlarged head portion (not shown, but similar to head portion 131 or head portion 446) that bears against an adjacent surface of the post 124 when the actuation member 502 is rotated. The threaded engagement of the threaded portion 508 of the actuation member 502 with a threaded portion of the frame (for example, a corresponding nut 127), brings the first post 122 closer to the second post, thereby radially expanding the frame.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Advantageously, prosthetic heart valves according to the examples previously discussed can reduce the bending stresses on the attached actuators by reducing the radial displacement between the end portions and the center portions of the actuators. In turn, this may mitigate the tendency of the actuators to bend or buckle during the implantation procedure, and reduce resulting impairment to the ability of the prosthetic heart valve to be radially expanded or contracted at the desired implantation site.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising a radially expandable frame comprising a plurality of interconnected struts, wherein the frame is radially expandable between a radially compressed state and a radially expanded state, a valvular structure disposed within frame and configured to regulate the flow of blood through the frame in one direction, and at least one an actuation assembly operatively coupled to the frame, wherein the actuation assembly comprises first and second bolts aligned end-to-end relative to each other, wherein an end portion of the first bolt is coupled to an adjacent end portion of the second bolt, wherein the first and second bolts are rotatable together in a first rotational direction to produce radial expansion of the frame from the radially compressed state to the radially expanded state.

Example 2. The prosthetic valve of any example herein, particularly example 1, wherein the second bolt comprises a threaded bore in the adjacent end portion of the second bolt, wherein a threaded section of the end portion of the first bolt is tightened into the threaded bore of the second bolt.

Example 3. The prosthetic valve of any example herein, particularly example 1, wherein a pin extends through the end portion of the first bolt and the adjacent end portion of the second bolt.

Example 4. The prosthetic valve of any example herein, particularly example 3, further comprising a second pin extending through the end portion of the first bolt and the adjacent end portion of the second bolt.

Example 5. The prosthetic valve of any example herein, particularly any of examples 1-4, wherein the frame comprises at least one vertically oriented post with a channel extending therethrough, and wherein the first bolt extends through the channel.

Example 6. The prosthetic valve of any example herein, particularly example 5, further comprising a washer disposed between the second bolt and the vertically oriented post, wherein the washer has an aperture and the end portion of the first bolt extends through the aperture.

Example 7. The prosthetic valve of any example herein, particularly example 6, wherein the washer further comprises an annular sleeve disposed around the aperture and extending axially into the channel.

Example 8. The prosthetic valve of any example herein, particularly any of examples 5-7, wherein the frame comprises at least a second vertically oriented post with a second channel extending therethrough, and wherein the second bolt extends through the second channel.

Example 9. The prosthetic valve of any example herein, particularly any of example 8, further comprising a threaded nut disposed within the second channel through which the second bolt extends.

Example 10. The prosthetic valve of any of any example herein, particularly any of examples 1-9, wherein the first bolt has a first diameter and the second bolt has a second diameter, wherein the second diameter is greater than the first diameter.

Example 11. The prosthetic valve of any example herein, particularly any of examples 1-10, wherein the diameter of the first bolt is less than or equal to 0.55 mm.

Example 12. The prosthetic valve of any example herein, particularly any of examples 1-11, wherein the diameter of the second bolt is greater than or equal to 0.6 mm.

Example 13. The prosthetic valve of any example herein, particularly any of examples 1-12, wherein the frame may be radially compressed to a diameter of less than 12 mm without either the first bolt or the second bolt buckling.

Example 14. The prosthetic valve of any example herein, particularly any of examples 1-13, wherein a head portion of the first bolt is configured to be releasably connected to a component of a delivery apparatus actuator.

Example 15. A medical assembly, comprising a prosthetic heart valve comprising a radially expandable frame comprising a plurality of interconnected struts, wherein the frame is radially expandable between a radially compressed state and a radially expanded state, a valvular structure disposed within frame and configured to regulate the flow of blood through the frame in one direction, and at least one an actuation assembly operatively coupled to the frame, and a delivery system configured to be releasably connected to a component of the actuation assembly and to advance the prosthetic heart valve through the vasculature of a patient, wherein the actuation assembly comprises first and second bolts aligned end-to-end relative to each other, wherein an end portion of the first bolt is coupled to an adjacent end portion of the second bolt, wherein the first and second bolts are rotatably coupled to rotate together in a first rotational direction to produce radial expansion of the frame from the radially compressed state to the radially expanded state, and wherein the first bolt has a first diameter and the second bolt has a second diameter, and wherein the first diameter is smaller than the second diameter.

Example 16. The medical assembly of any example herein, particularly example 15, wherein the second bolt comprises a threaded bore in the adjacent end portion of the second bolt, wherein a threaded section of the end portion of the first bolt is tightened into the threaded bore of the second bolt.

Example 17. The medical assembly of any example herein, particularly example 15, wherein a pin extends through an end portion of the first bolt and the adjacent end portion of the second bolt.

Example 18. The medical assembly of any example herein, particularly example 17, further comprising a second pin extending through the end portion of the first bolt and the adjacent end portion of the second bolt.

Example 19. The medical assembly of any example herein, particularly any of examples 15-18, wherein the first bolt extends through a bore in at least one vertically oriented post.

Example 20. The medical assembly of any example herein, particularly example 19, further comprising a washer disposed between a head portion of the second bolt and a distal end of the vertically oriented post, wherein the first bolt extends through an aperture of the washer.

Example 21. The actuation assembly of any example herein, particularly example 20, wherein the washer further comprises an annular sleeve portion extending axially into the bore of the vertically oriented post.

Example 22. The actuation assembly of any example herein, particularly any of examples 15-21, wherein the second bolt extends through a bore in a second vertically oriented post.

Example 23. The medical assembly of any example herein, particularly example 22, further comprising a threaded nut coupled to the second vertically oriented post through which the second bolt extends.

Example 24. The actuation assembly of any example herein, particularly any of examples 15-23, wherein the diameter of the first bolt is less than or equal to 0.55 mm.

Example 25. The actuation assembly of any example herein, particularly any of examples 15-23, wherein the diameter of the second bolt is greater than or equal to 0.6 mm.

Example 26. A prosthetic heart valve, comprising a radially expandable frame having a first end, a second end, and a longitudinal axis extending between the first end and the second end, wherein the frame is radially expandable between a radially compressed state and a radially expanded state, a valvular structure disposed within frame and configured to regulate the flow of blood through the frame in one direction, and at least one actuation assembly operatively coupled to the frame, wherein the frame comprises a plurality of axial posts extending parallel to the longitudinal axis and a plurality of angled struts arranged in circumferentially extending rows of angled struts, and wherein the actuation assembly comprises first and second bolts aligned end-to-end relative to each other, wherein an end portion of the first bolt is coupled to an adjacent end portion of the second bolt, wherein the first and second bolts are rotatable together in a first rotational direction to produce radial expansion of the frame from the radially compressed state to the radially expanded state, and wherein the first bolt extends through a channel in a first axial post of the plurality of axial posts.

Example 27. The prosthetic heart valve of any example herein, particularly example 26, wherein the second bolt extends through a channel in a second axial post of the plurality of axial posts.

Example 28. The prosthetic heart valve of any example herein, particularly any of examples 26-27, wherein the actuation assembly further comprises a threaded nut disposed within a window in the second axial post, wherein the second bolt extends through the nut.

Example 29. The prosthetic heart valve of any example herein, particularly any of examples 25-28, wherein the adjacent end portion of the second bolt comprises a threaded bore, wherein a threaded section of a distal end portion of the first bolt is tightened into the threaded bore of the second bolt.

Example 30. The prosthetic heart valve of any example herein, particularly any of examples 25-29, wherein the actuation assembly further comprises a first pin extending through the end portion of the first bolt and the adjacent end portion of the second bolt.

Example 31. The prosthetic heart valve of any example herein, particularly example 30, wherein the actuation assembly further comprises a second pin extending through the end portion of the first bolt and the adjacent end portion of the second bolt.

Example 32. The prosthetic heart valve of any example herein, particularly any of examples 26-31, further comprising a washer disposed between the adjacent end portion of the second bolt and the first axial post, wherein the washer comprises an aperture through which the first bolt extends.

Example 33. The prosthetic heart valve of any example herein, particularly example 32, wherein the washer further comprises an annular sleeve portion extending axially into the channel in the first axial post.

Example 34. The prosthetic heart valve of any example herein, particularly any of examples 26-33, wherein the first bolt has a first diameter and the second bolt has a second diameter larger than the first diameter.

Example 35. The prosthetic heart valve of any of any example herein, particularly any of examples 26-34, wherein the first bolt and the second bolt are rotatable together in a second rotational direction opposite to the first rotational direction to produce radial compression of the frame from the radially expanded state to a radially compressed state or to a partially radially expanded state.

Example 36. The prosthetic heart valve of any example herein, particularly any of examples 25-35, wherein the diameter of the first bolt is less than or equal to 0.55 mm.

Example The prosthetic heart valve of any example herein, particularly any of examples 25-36, wherein the diameter of the second bolt is greater than or equal to 0.6 mm.

Example 38. The prosthetic heart valve of any example herein, particularly example 25-37, wherein a proximal end portion of the first bolt is configured to be releasably attached to a component of a delivery apparatus.

Example 39. The prosthetic heart valve of any example herein, particularly example 25-37, further comprising a skirt disposed around and attached to the exterior of the frame.

Example 40. A prosthetic heart valve, comprising a radially expandable frame comprising a plurality of interconnected struts, wherein the frame is radially expandable between a radially compressed state and a radially expanded state, a valvular structure disposed within frame and configured to regulate the flow of blood through the frame in one direction, at least one an actuation assembly operatively coupled to the frame, at least one nut coupled to the actuation assembly, and wherein the actuation assembly comprises first and second bolts aligned end-to-end relative to each other, wherein an end portion of the first bolt is coupled to an adjacent end portion of the second bolt, wherein the second bolt comprises a threaded portion that extends through and engages internal threads of the nut, wherein the first and second bolts are rotatable together in a first rotational direction to produce radial expansion of the frame from the radially compressed state to the radially expanded state.

Example 41. The prosthetic heart valve of any example herein, particularly example 40, further comprising a bore in one strut of the plurality of interconnected struts, which receives a component of the actuation assembly.

Example 42. The prosthetic heart valve of any example herein, particularly example 41, wherein the bore further comprises a window configured to receive the nut.

Example 43. The prosthetic heart valve of any example herein, particularly example 40-42, wherein the second bolt comprises a threaded bore in the adjacent end portion of the second bolt, wherein a threaded section of the end portion of the first bolt is tightened into the threaded bore of the second bolt.

Example 44. The prosthetic heart valve of any example herein, particularly example 40-42, wherein a pin extends through an end portion of the first bolt and the adjacent end portion of the second bolt.

Example 45. The prosthetic heart valve of any example herein, particularly example 44, further comprising a second pin extending through the end portion of the first bolt and the adjacent end portion of the second bolt.

Example 46. The prosthetic heart valve of any example herein, particularly any of examples 40-45, wherein the frame comprises a first axially oriented post and a second axially oriented post, wherein the first axially oriented post and the second axially oriented post are axially aligned with each other, wherein a bore extends axially through both the first axially oriented post and the second axially oriented post and receives the first and second bolts.

Example 47. The prosthetic heart valve any example herein, particularly example 46, further comprising a washer disposed between the adjacent end portion of the second bolt and a distal end portion of the first axially oriented post.

Example The prosthetic heart valve of any example herein, particularly example 47, wherein the washer comprises an aperture and the first bolt extends through the aperture.

Example 49. The prosthetic heart valve of any example herein, particularly any of examples 47-48, wherein the washer comprises an annular sleeve portion extending axially into the bore in the first axially oriented post.

Example 50. The prosthetic heart valve of any example herein, particularly any of examples 40-49, wherein the first bolt has a first diameter, the second bolt has a second diameter, and the second diameter is greater than the first diameter.

Example 51. The prosthetic heart valve of any example herein, particularly any of examples 40-50, wherein the diameter of the first bolt is less than or equal to 0.55 mm.

Example 52. The prosthetic heart valve of any example herein, particularly any of examples 40-51, wherein the diameter of the second bolt is greater than or equal to 0.6 mm.

Example 53. The prosthetic heart valve of any example herein, particularly any of examples 40-52, wherein the first bolt comprises a head portion and the head portion is configured to be releasably connected to a component of a delivery apparatus actuator.

Example 54. A prosthetic heart valve, comprising a frame having a first end, a second end, and a longitudinal axis extending between the first end and the second end, wherein the frame is radially expandable between a radially compressed state and a radially expanded state, and wherein the frame comprises a plurality of vertical posts extending parallel to the longitudinal axis and a plurality of angled struts arranged in circumferentially extending rows of angled struts, a valvular structure disposed within frame and configured to regulate the flow of blood through the frame in one direction, at least one actuation assembly comprising a first bolt and a second bolt aligned end-to-end, wherein the first bolt extends through a channel in a first vertical post of the plurality of vertical post wherein an end portion of the first bolt is coupled to an adjacent end portion of the second bolt, and wherein the first and second bolts are rotatable together in a first rotational direction to produce radial expansion of the frame from the radially compressed state to the radially expanded state, and a washer disposed between the adjacent end portion of the second bolt and the first vertical post.

Example 55. The prosthetic heart valve of any example herein, particularly example 54, wherein the washer comprises an aperture and the first bolt extends through the aperture.

Example 56. The prosthetic heart valve of any example herein, particularly any of examples 54-55, wherein the washer comprises an annular sleeve portion extending axially into the channel in the first vertical post.

Example 57. The prosthetic heart valve of any example herein, particularly any of examples 54-56, wherein the second bolt extends through a second channel in a second vertical post of the plurality of vertical posts.

Example The prosthetic heart valve of any example herein, particularly any of examples 54-57, wherein the second channel further comprises an opening that receives a nut through which the second bolt extends.

Example 59. The prosthetic heart valve of any example herein, particularly any of examples 54-58, wherein the second bolt comprises a threaded bore in the adjacent end portion of the second bolt, wherein a threaded section of the end portion of the first bolt is tightened into the threaded bore of the second bolt.

Example 60. The prosthetic heart valve of any example herein, particularly any of examples 54-59, wherein a pin extends through an end portion of the first bolt and the adjacent end portion of the second bolt.

Example 61. The prosthetic heart valve of any example herein, particularly example 60, further comprising a second pin extending through the end portion of the first bolt and the adjacent end portion of the second bolt.

Example 62. The prosthetic heart valve of a any example herein, particularly any of examples 54-61, wherein the first bolt has a first diameter and the second bolt has a second diameter larger than the first diameter.

Example 63. The prosthetic heart valve of any example herein, particularly any of examples 54-62, wherein the first bolt comprises a head portion and the head portion is configured to be releasably connected to a component of a delivery apparatus actuator.

Example 64. A prosthetic valve, comprising a radially expandable frame comprising a plurality of interconnected struts, wherein the frame is radially expandable between a radially compressed state and a radially expanded state, a valvular structure disposed within frame and configured to regulate the flow of blood through the frame in one direction, and at least one an actuation assembly operatively coupled to the frame, wherein the actuation assembly comprises first and second actuation members aligned end-to-end relative to each other, wherein an end portion of the first actuation member is coupled to an adjacent end portion of the second actuation member, wherein the first and second actuation members are rotatable together in a first rotational direction to produce radial expansion of the frame from the radially compressed state to the radially expanded state.

Example 65. The prosthetic valve of any example herein, particularly example 64, wherein the second actuation member comprises a threaded bore in the adjacent end portion of the second actuation member, wherein a threaded section of the end portion of the first actuation member is tightened into the threaded bore of the second actuation member.

Example 66. The prosthetic valve of any example herein, particularly example 64, wherein a pin extends through the end portion of the first actuation member and the adjacent end portion of the second actuation member.

Example 67. The prosthetic valve of any example herein, particularly example 66, further comprising a second pin extending through the end portion of the first actuation member and the adjacent end portion of the second actuation member.

Example 68. The prosthetic valve of any example herein, particularly any of examples 64-67, wherein the frame comprises at least one vertically oriented post with a channel extending therethrough, and wherein the first actuation member extends through the channel.

Example 69. The prosthetic valve of any example herein, particularly example 68, further comprising a washer disposed between the second actuation member and the vertically oriented post, wherein the washer has an aperture and the end portion of the first actuation extends through the aperture.

Example 70. The prosthetic valve of any example herein, particularly example 69, wherein the washer further comprises an annular sleeve disposed around the aperture and extending axially into the channel.

Example 71. The prosthetic valve of any example herein, particularly any of examples 68-70, wherein the frame comprises at least a second vertically oriented post with a second channel extending therethrough, and wherein the second actuation member extends through the second channel.

Example 72. The prosthetic valve of any example herein, particularly example 71, further comprising a threaded nut disposed within the second channel through which the second actuation member extends.

Example 73. The prosthetic valve of any example herein, particularly any of examples 64-72, wherein the first actuation member has a first diameter and the second actuation member has a second diameter, wherein the second diameter is greater than the first diameter.

Example 74. The prosthetic valve of any example herein, particularly any of examples 64-73, wherein the diameter of the first actuation member is less than or equal to 0.55 mm.

Example 75. The prosthetic valve of any example herein, particularly any of examples 64-74, wherein the diameter of the second actuation member is greater than or equal to 0.6 mm.

Example 76. The prosthetic valve of any example herein, particularly any of examples 64-75, wherein the frame may be radially compressed to a diameter of less than 12 mm without either the first actuation member or the second actuation member buckling.

Example 77. The prosthetic valve of any example herein, particularly any of examples 64-76, wherein a head portion of the first actuation member is configured to be releasably connected to a component of a delivery apparatus actuator.

Example 78. A prosthetic valve, comprising a radially expandable frame comprising a first end portion, a second end portion, and a plurality of interconnected struts, wherein the frame is radially expandable between a radially compressed state and a radially expanded state; a valvular structure disposed within frame and configured to permit the flow of blood from the first end portion to the second end portion of the frame and to prevent the flow of blood from the second end portion to the first end portion of the frame; and an actuation assembly operatively coupled to the frame, the actuation assembly comprising a stopper having an aperture, an actuation member extending through the aperture in the stopper and a securing pin protruding from at least one side of the actuation member; wherein the securing pin limits motion of the stopper relative to the actuation member in a first axial direction, and wherein the actuation member is rotatable in a first direction to produce radial expansion of the frame from a radially compressed state to a radially expanded state and rotatable in a second rotational direction to produce radial compression of the frame from the radially expanded state to the radially compressed state.

Example 79. The prosthetic valve of any example herein, particularly example 78, wherein the securing pin is positioned further towards the first end portion of the frame than the stopper.

Example 80. The prosthetic valve of any example herein, particularly example 78, wherein the securing pin is positioned further towards the second end portion of the frame than the stopper.

Example 81. The prosthetic valve of any example herein, particularly any of examples 78-80, wherein the frame comprises at least one axially extending first post and at least one axially extending second post arranged as a pair of axially aligned vertical posts, and wherein the actuation assembly extends between and interconnects the first post and the second post.

Example 82. The prosthetic valve of any example herein, particularly example 81, wherein the first post comprises a first axial bore and the second post comprises a second axial bore, wherein the first axial bore and the second axial bore are dimensioned to receive portions of the at least one actuation assembly.

Example 83. The prosthetic valve of any example herein, particularly any of examples 81-82, wherein the stopper is positioned between the first post and the second post.

Example 84. The prosthetic valve of any example herein, particularly any of examples 78-83, wherein the securing pin has a length, the stopper has an outer diameter, and the length of the pin is less than the outer diameter of the stopper.

Example 85. The prosthetic valve of any example herein, particularly example 84, wherein the outer diameter of the stopper is 0.8 mm or less.

Example 86. The prosthetic valve of any example herein, particularly any of examples 78-85, wherein the stopper has a height that is 0.5 mm or less.

Example 87. The prosthetic valve of any example herein, particularly any of examples 78-86, wherein the stopper comprises a biocompatible material.

Example 88. The prosthetic valve of any example herein, particularly example 87, wherein the biocompatible material is a cobalt-chromium alloy.

Example 89. The prosthetic valve of any example herein, particularly example 87, wherein the biocompatible material comprises polyether ether ketone, teflon, polyethylenimine, or a combination thereof.

Example 90. The prosthetic valve of any example herein, particularly any of examples 78-89, wherein the securing pin has a diameter ranging from 0.2 mm to 0.4 mm.

Example 91. The prosthetic valve of any example herein, particularly any of examples 78-90, wherein rotation of the actuation member in one direction produces translation in a second axial direction opposite the first axial direction with the securing pin bearing against the stopper and urging the stopper to translate relative to the actuation member.

Example 92. The prosthetic valve of any example herein, particularly any of examples 78-91, wherein the actuation member comprises a head portion that is configured to be releasably connected to a component of a delivery apparatus actuator.

Example 93. The prosthetic valve of any example herein, particularly example 92, wherein the head portion is driveable in rotation to extend and retract threadedly engaged components of the actuation assembly to radially expand and radially compress the frame.

Example 94. The prosthetic valve of any example herein, particularly any of examples 78-93, wherein the prosthetic heart valve comprises two or more actuation assemblies, each operatively coupled to the frame and rotatable in a first rotational direction to produce radial expansion of the frame from a radially compressed state to a radially expanded state and rotatable in a second rotational direction to produce radial compression of the frame from the radially expanded state to the radially compressed state.

Example 95. A prosthetic valve, comprising a radially expandable frame comprising a vertically oriented first strut and a vertically oriented second strut, wherein the frame is radially expandable between a radially compressed state and a radially expanded state; a valvular structure disposed within the frame and configured to regulate the flow of blood through the frame in one direction; and at least one an actuation assembly operatively coupled to the frame, comprising an actuation member, a stopper positioned on the actuation member, and a protrusion extending from an outer surface of the actuation member; wherein bores in the first strut and in the second strut are dimensioned to receive portions of the actuation member; wherein the stopper is positioned between the first strut and the second strut, and the axial movement of the stopper along the actuation member is limited in at least one direction by the protrusion; and wherein the actuation member is rotatable in a first direction to move axially with the protrusion contacting the stopper to urge the first strut away from the second strut, thereby radially compressing the frame.

Example 96. The prosthetic valve of any example herein, particularly example 95, wherein the first strut comprises a window, the actuation assembly further comprises a nut positioned in the window and the actuation member comprises an externally threaded end portion that engages with the nut.

Example 97. The prosthetic valve of any example herein, particularly any of examples 95-96, wherein the actuation member comprises a lateral bore to receive the securing pin.

Example 98. The prosthetic valve of any example herein, particularly any of examples 95-97, wherein the protrusion is configured to withstand an axially oriented force of at least 40 N.

Example 99. The prosthetic valve of any example herein, particularly any of examples 95-98, wherein the actuation member is configured to withstand a tensile stress of at least 400 MPa.

Example 100. The prosthetic valve of any example herein, particularly any of examples 95-99, wherein the protrusion comprises a biocompatible material.

Example 101. The prosthetic valve of any example herein, particularly example 100, wherein the biocompatible material is biocompatible metal alloy or biocompatible polymer.

Example 102. The prosthetic valve of any example herein, particularly any of examples 95-101, wherein the protrusion is positioned between the stopper and the first post.

Example 103. The prosthetic valve of any example herein, particularly any of examples 95-102, wherein the actuation member comprises a head portion that is configured to be releasably connected to a component of a delivery apparatus actuator.

Example 104. The prosthetic valve of any example herein, particularly any of examples 95-103, wherein the prosthetic valve comprises two or more actuation assemblies, each operatively coupled to the frame and rotatable in a first rotational direction to produce radial expansion of the frame from a radially compressed state to a radially expanded state and rotatable in a second rotational direction to produce radial compression of the frame from the radially expanded state to the radially compressed state.

Example 105. A medical assembly, comprising a radially expandable frame having a first frame end portion, a second frame end portion, and a longitudinal axis extending between the first end and the second end; and at least one actuation assembly extending longitudinally from the first frame end portion towards the second frame end portion to interconnect first and second portions of the frame, the actuation assembly comprising an actuation member having a first end portion with a head portion adjacent the first frame end and an externally threaded second end portion, a stopper disposed around the actuation member, and a securing pin extending through the actuation member transverse to the longitudinal axis; wherein the securing pin is configured to limit the movement of the stopper along the longitudinal axis in at least one direction; wherein the stopper is configured to limit the movement of the actuation member along the longitudinal axis in at least one direction; and wherein the actuation member is rotatable in a first rotational direction to produce radial expansion of the frame from a radially compressed state to a radially expanded state and rotatable in a second rotational direction to produce radial compression of the frame from the radially expanded state to the radially compressed state.

Example 106. The medical assembly of any example herein, particularly example 105, wherein the first end portion of the frame comprises a first post extending along the longitudinal axis from the first end portion towards the second end portion and the second end portion of the frame comprises a second post extending from the second end portion towards the first end portion of the frame, and wherein the actuation member has an exposed segment extending between the first post and the second post.

Example 107. The medical assembly of any example herein, particularly example 106, wherein the stopper is positioned between the first post and the second post and configured to contact an end portion of the second post as the frame is radially compressed from the radially expanded state to the radially compressed state.

Example 108. The medical assembly of any example herein, particularly example 107, wherein the securing pin is positioned between the stopper and the first post.

Example 109. The medical assembly of any example herein, particularly any of examples 105-108, wherein the securing pin extends through a bore in the actuation member.

Example 110. The medical assembly of any example herein, particularly any of examples 105-108, wherein the securing pin is integrally formed with the actuation member.

Example 111. The medical assembly of any example herein, particularly any of examples 105-110, wherein the head portion is releasably connectable to a component of a delivery apparatus actuator.

Example 112. The medical assembly of any example herein, particularly any of examples 105-111, further comprising a valvular structure disposed within frame and configured to permit the flow of blood along the longitudinal axis in a first axial direction and to restrict the flow of blood along the longitudinal axis in a second axial direction opposite to the first axial direction.

Example 113. The medical assembly, frame, prosthetic heart valve, valvular structure, or delivery device of any example herein, particularly any of examples 1-112, wherein the medical assembly, frame, prosthetic heart valve, valvular structure, or delivery device is sterilized.

In view of the many possible embodiments to which the principles of the disclosure may be applied, it should be recognized that the illustrated configurations depict examples of the disclosed technology and should not be taken as limiting the scope of the disclosure or the claims. Rather, the scope of the claimed subject matter is defined by the following claims and their equivalents.

1. A prosthetic valve, comprising:
a radially expandable frame comprising a plurality of interconnected struts, wherein the frame is radially expandable between a radially compressed state and a radially expanded state;
a valvular structure disposed within frame and configured to regulate the flow of blood through the frame in one direction; and
at least one an actuation assembly operatively coupled to the frame, wherein the actuation assembly comprises first and second bolts aligned end-to-end relative to each other, wherein an end portion of the first bolt is coupled to an adjacent end portion of the second bolt, wherein the first and second bolts are rotatable together in a first rotational direction to produce radial expansion of the frame from the radially compressed state to the radially expanded state.

2. The prosthetic valve of claim 1, wherein the second bolt comprises a threaded bore in the adjacent end portion of the second bolt, wherein a threaded section of the end portion of the first bolt is tightened into the threaded bore of the second bolt.

3. The prosthetic valve of claim 1, wherein a pin extends through the end portion of the first bolt and the adjacent end portion of the second bolt.

4. The prosthetic valve of claim 1, wherein the frame comprises at least one vertically oriented post with a channel extending therethrough, and wherein the first bolt extends through the channel.

5. The prosthetic valve of claim 4, further comprising a washer disposed between the second bolt and the vertically oriented post, wherein the washer has an aperture and the end portion of the first bolt extends through the aperture.

6. The prosthetic valve of claim 5, wherein the washer further comprises an annular sleeve disposed around the aperture and extending axially into the channel.

7. The prosthetic valve of claim 1, wherein the first bolt has a first diameter and the second bolt has a second diameter, wherein the second diameter is greater than the first diameter.

8. The prosthetic valve of claim 1, wherein the diameter of the first bolt is less than or equal to 0.55 mm.

9. The prosthetic valve of claim 1, wherein the diameter of the second bolt is greater than or equal to 0.6 mm.

10. The prosthetic valve of claim 1, wherein a head portion of the first bolt is configured to be releasably connected to a component of a delivery apparatus actuator.

11. A prosthetic heart valve, comprising
a radially expandable frame comprising a plurality of interconnected struts, wherein the frame is radially expandable between a radially compressed state and a radially expanded state;
a valvular structure disposed within frame and configured to regulate the flow of blood through the frame in one direction;
at least one an actuation assembly operatively coupled to the frame; and
at least one nut coupled to the actuation assembly; and
wherein the actuation assembly comprises first and second bolts aligned end-to-end relative to each other, wherein an end portion of the first bolt is coupled to an adjacent end portion of the second bolt, wherein the second bolt comprises a threaded portion that extends through and engages internal threads of the nut, wherein the first and second bolts are rotatable together in a first rotational direction to produce radial expansion of the frame from the radially compressed state to the radially expanded state.

12. The prosthetic heart valve of claim 11, further comprising a bore in one strut of the plurality of interconnected struts, which receives a component of the actuation assembly.

13. The prosthetic heart valve of claim 12, wherein the bore further comprises a window configured to receive the nut.

14. The prosthetic heart valve of claim 11, wherein a pin extends through an end portion of the first bolt and the adjacent end portion of the second bolt.

15. The prosthetic heart valve of claim 11, wherein the frame comprises a first axially oriented post and a second axially oriented post, wherein the first axially oriented post and the second axially oriented post are axially aligned with each other, wherein a bore extends axially through both the first axially oriented post and the second axially oriented post and receives the first and second bolts.

16. The prosthetic heart valve of claim 11, wherein the first bolt has a first diameter, the second bolt has a second diameter, and the second diameter is greater than the first diameter.

17. A prosthetic heart valve, comprising:
a frame having a first end, a second end, and a longitudinal axis extending between the first end and the second end, wherein the frame is radially expandable between a radially compressed state and a radially expanded state, and wherein the frame comprises a plurality of vertical posts extending parallel to the longitudinal axis and a plurality of angled struts arranged in circumferentially extending rows of angled struts;
a valvular structure disposed within frame and configured to regulate the flow of blood through the frame in one direction;
at least one actuation assembly comprising a first bolt and a second bolt aligned end-to-end, wherein the first bolt extends through a channel in a first vertical post of the plurality of vertical post wherein an end portion of the first bolt is coupled to an adjacent end portion of the second bolt, and wherein the first and second bolts are rotatable together in a first rotational direction to produce radial expansion of the frame from the radially compressed state to the radially expanded state; and
a washer disposed between the adjacent end portion of the second bolt and the first vertical post.

18. The prosthetic heart valve of claim 17, wherein the washer comprises an annular sleeve portion extending axially into the channel in the first vertical post.

19. The prosthetic heart valve of claim 17, wherein the second bolt comprises a threaded bore in the adjacent end portion of the second bolt, wherein a threaded section of the end portion of the first bolt is tightened into the threaded bore of the second bolt.

20. The prosthetic heart valve of claim 17, wherein the first bolt has a first diameter and the second bolt has a second diameter larger than the first diameter.
